(19) United States
(12) Patent Application Publication
(10) Pub. No.: US 2022/0351354 A1
(43) Pub. Date: Nov. 3, 2022

(54) METHOD OF PRODUCING 3D TOMOSYNTHESIS IMAGES OF A COMPOSITE MATERIAL

(71) Applicant: Adaptix Ltd., Begbroke (GB)

(72) Inventors: Gil Travish, Begbroke (GB); Mark Evans, Begbroke (GB)

(21) Appl. No.: 17/859,683

(22) Filed: Jul. 7, 2022

Related U.S. Application Data
(63) Continuation of application No. PCT/GB2020/053246, filed on Dec. 16, 2020.

(30) Foreign Application Priority Data
Jan. 7, 2020 (GB) 2000156.6

(51) Int. Cl. G06T 7/00; G06T 7/70; G01N 23/044; G06F 16/51; G06Q 30/00

(52) U.S. Cl. CPC G06T 7/001; G06T 7/70; G01N 23/044; G06F 16/51; G06Q 30/0185; G06T 2207/30204; G06T 2207/10116; G06T 2200/04; G06T 2207/30108; G06T 2207/10012; G01N 2223/417

(57) ABSTRACT

To identify and/or assess structural integrity of a composite material comprising fiduciary markers which attenuate x-rays to an extent greater than the rest of the material, a method is provided wherein x-ray 3D tomosynthesis images of the composite material are created using an array of x-ray emitters and a digital x-ray detector wherein the array of x-ray emitters and the digital x-ray detector are maintained in fixed relation to one another and to the composite material, the 3D tomosynthesis images being used to determine the relative location of at least some of the fiduciary markers with respect to one another; a database is provided for storing the relative location of at least some of the fiduciary markers with respect to one another, further x-ray 3D tomosynthesis images of the same, or a different, composite material may be checked against the data in the database to ascertain structural integrity and/or identity of the material.

### RELATED APPLICATIONS

This application claims the benefit under 35 U.S.C. § 120, and is a continuation, of co-pending International Application PCT/GB2020/053246, filed Dec. 16, 2020 and designating the US, which claims priority to GB Application 2000156.6, filed Jan. 7, 2020, such GB Application also being claimed priority to under 35 U.S.C. § 119. These GB and International applications are incorporated by reference herein in their entireties.

### FIELD

The present invention relates generally to a method of obtaining x-ray images and apparatus arranged to operate according to the method and finds particular, although not exclusive, utility in reducing dosage without undue loss of clarity in the images.

### BACKGROUND

Composite materials are generally defined as consisting of two or more materials, combined in such a way that the composite's properties are distinct from those of the individual materials. Common examples include fiber-reinforced plastics and carbon fiber but can also include plastic-metal laminates and other laminates or matrix materials.

Non-destructive evaluation and testing of components and particularly of components containing composites is challenging. For example, delamination is a mode of failure where a material fractures into layers. A variety of materials including laminate composites can fail by delamination.

Structural Health Monitoring (SHM) may be defined as the “acquisition, validation and analysis of technical data to facilitate life-cycle management decisions.” More generally, SHM denotes a reliable system with the ability to detect and interpret adverse “changes” in a structure due to damage or normal operation.

SHM is more advantageous to some industries, such as the aerospace industry, since damage can lead to catastrophic (and expensive) failures, and the vehicles involved have regular costly inspections. Aircraft are increasingly including composite materials to take advantage of their excellent specific strength and stiffness properties, as well their ability to reduce radar cross-section and “part-count”. The disadvantage, however, is that composite materials present challenges for design, maintenance and repair over metallic parts since they tend to fail by distributed and interacting damage modes. Furthermore, damage detection in composites is much more difficult due to the anisotropy of the material, the conductivity of the fibers, the insulative properties of the matrix, and the fact that much of the damage often occurs beneath the top surface of the laminate, for instance with barely visible impact damage.

Currently successful composite non-destructive testing techniques for small laboratory specimens, such as radiographic detection (penetrant enhanced X-ray) and hydro-ultrasonics (C-scan), are impractical for large components and integrated vehicles.

Furthermore, the main limitation of current visualization techniques is a very limited possibility to image so-called closed delamination in which delaminated layers are in contact practically with no physical gap.

Several techniques have been researched for detecting damage in composite materials focused on modal response. These methods are among the earliest and most common, principally because they are simple to implement on any size structure. Structures can be excited by ambient energy, an external shaker or embedded actuators, and embedded strain gauges, piezometers or accelerometers can be used to monitor the structural dynamic responses. Changes in normal vibrational modes can be correlated to loss of stiffness in a structure, and usually analytical models or experimentally determined response-history tables are used to predict the corresponding location of damage. The difficulty, however, comes in the interpretation of the data collected by this type of system. There are also detection limitations imposed by the resolution and range of the individual sensors chosen, and the density with which they are distributed over the structure.

Another area of interest is that of 3D printing, or additive manufacturing, where often a single material is applied, layer by layer, to build up an object. While conventional 3D printing may not be considered a composite in the traditional sense, the layered structure has similar challenges to laminates in that they have low x-ray contrast, can suffer from hidden voids and flaws.

### SUMMARY

A problem with such products is that of “ply wrinkling” with various causes including thermal history, shifting of the vacuum bag, non-uniform resin, etc. These wrinkles may render a part unfit, but such wrinkling may go undetected until late in the manufacturing process (adding significant costs to the cast-off part) or entirely undetected (leading to an unsuitable part being deployed in the field). Therefore, detection of such wrinkles and related defects during manufacture is of key interest.

Ultrasound provides limited information about structural integrity of many types of parts and can easily fail in complex assemblies. Two-dimension x-rays do not reveal flaws in structures with complex overlying and underlying layers. Existing 3D x-ray imaging (i.e. CT) can be slow, expensive, heavy and very complex to field as it requires three-phase power and a radiation shielded room. Also, CT typically use high doses of radiation which may damage some sensitive components. Conventional mechanical tests (strain gauges, magnaflux, etc.) often do not work well with additive manufacturing and can fail to reveal hidden flaws until failure occurs.

At the same time, counterfeit components present a serious concern. Counterfeit products are now a common occurrence which can lead to safety concerns. There is clearly a need to identify counterfeit products.

There is therefore a need for a composite material which can be checked for structural integrity and/or its identity in a non-destructive manner, and for a method of checking said structural integrity and/or identity.

In a first aspect, the invention provides a method of producing 3D tomosynthesis images of at least a portion of a composite material, the composite material including fiber mixed with resinous material, and a plurality of fiduciary markers, the fiduciary markers comprising elements which attenuate x-rays to an extent greater than the fiber and resinous material such that their location within the portion of composite material is determinable by means of x-ray imaging, the method comprising the step of providing a composite material, providing an array of x-ray emitters and a digital x-ray detector wherein the array of x-ray emitters and the digital x-ray detector are maintained in fixed relation to one another and to the composite material, x-ray imaging at least a portion of the composite material to provide a first set of 3D tomosynthesis images to determine the relative location of at least some of the fiduciary markers with respect to one another, providing a database and storing the relative location of at least some of the fiduciary markers with respect to one another in the database.

In this way, a 3D tomosynthesis model may be created which may be stored electronically, in the database, and which may be interrogated/processed in the future to provide the locations of at least some of the fiduciary markers. The locations of the markers may be relative to other markers or a datum such as a particular identified point within, or on the surface of, the composite material. The information may be considered to be a map.

The method may further comprise the step of comparing the relative locations of the at least some of the fiduciary markers with a predetermined set of locations to evaluate the quality of the composite material. The predetermined set of locations may be stored in the database.

For instance, if the composite material is constructed in a particular predetermined manner and with the fiduciary markers being added to the resinous material at predetermined locations then the relative location of the markers should match with a standard, saved, set of data. However, if a comparison indicates that the locations are different, or at least the difference exceeds a predetermined threshold, it may be because of errors in the manufacturing process. This may help identify products which do not meet quality control standards.

The method may further comprise the step of x-ray imaging the portion of composite material at a point in time after the initial imaging to provide a second set of 3D tomosynthesis images to determine the relative location of at least some of the fiduciary markers with respect to one another; and may compare the relative locations of the fiduciary markers in the first and second sets of 3D tomosynthesis images to evaluate the occurrence of change in the structural integrity of the portion of composite material.

The second set of images may include all, or only some, of the markers in the first set of images. The step of comparison may include the step of interrogating the database.

In this way, the structural health of the composite material may be monitored over time. For instance, if the relative locations, when compared, are different, or exceed a threshold, it may indicate failure of the material through such means as delamination. This may help identify products which need replacing or repair before they fail and cause subsequent problems.

The method may further comprise the step of x-ray imaging at least a portion of another composite material to provide another set of 3D tomosynthesis images to determine the relative location of at least some of the fiduciary markers with respect to one another; and may compare the relative locations of the fiduciary markers in the first set and other set of 3D tomosynthesis images to evaluate the identity of the other composite material.

In this way, the relative location of markers in one material may be compared to the relative location of the markers in the first set. The first set may be considered to be the standard against which other products are compared. If the relative locations match, or at least within a predetermined tolerance, the second, other composite material may be determined to have been manufactured in the same manner as the first composite material. This may allow identification of manufacturing methods and/or manufacturing locations, such that the step of evaluating the identity of the other composite material includes the step of determining if the other composite material is a counterfeit product.

The step of evaluating the identity of the other composite material may include the step of interrogating the database. Subscribers to the database may use it to verify component products as not being counterfeit.

The method may further comprise the step of providing 2D x-ray imaging apparatus and x-ray imaging at least a portion of the composite material to provide a 2D x-ray image to determine the relative location of at least some of the fiduciary markers with respect to one another; and may compare the relative locations of the fiduciary markers in the 2D image with the first set of images to evaluate the identity of the other composite material.

In this regard, it may be possible for even a 2D image showing locations of fiduciary markers to provide enough information for the identity of the material to be ascertained or verified. In this manner, the full 3D image of the standard material, against which the 2D image is compared, may be maintained confidential, from subscribers to the database, for instance. This may allow the step of evaluating the identity of the other composite material to include the step of determining if the other composite material is a counterfeit product.

The step of evaluating the identity of the other composite material may include the step of interrogating the database.

The method may further include the step of providing a processor and using the processor to determine the relative location of the at least some of the fiduciary markers with respect to one another. In this regard, it is to be understood that a processor may be used to process the raw information received from the detector to create the necessary data. A processor may also be used to produce the tomosynthesis images. A processor may also be used to compare the relative locations of the markers between sets of images to evaluate different materials and compare them against other materials and data sets stored in the database so as to evaluate the structural integrity of materials and/or the identity thereof.

The method may further include the step of repeatedly moving either or both of the array of x-ray emitters and the digital x-ray detector to a different portion of the composite material for x-ray imaging thereof, so as to x-ray image multiple portions of a composite material, wherein the array of x-ray emitters and the digital x-ray detector are maintained in fixed relation to one another and to the composite material at the time of x-ray imaging.

In this way, a large object comprising composite material, such as an aircraft wing, may be imaged in portions of relatively small areas, over time, by moving the array and detector to a different place each time, such that the entire object is imaged.

The method may further include the step of processing the various sets of x-ray images obtained for each portion of the composite material to create a single set of contiguous images of the composite material.

Any comparison of images may be undertaken through pattern analysis such that it is at least a portion of the patterns of markers within the various (such as the first and second sets of) images which are compared.

The term “composite material” may include any one or more of a composite material, a laminate material, a matrix material and other similar materials comprising more elements having different physical properties. It may be defined as consisting of two or more materials, combined in such a way that the composite material's properties are distinct from those of the individual materials. Common examples include fiber-reinforced plastics but can also include plastic-metal laminates and other laminates or matrix materials. A composite material may include 3D printed/additive manufactured products.

The term “fiber” may include any one or more of carbon fiber, fiber, fiber reinforced material, woven fiber, non-woven fiber. The term fiber may comprise Kevlar (®), viscose, Tencel (®), Rayon (®), and other polymers.

The term “resinous material” may include any one or more of a filler, resin, epoxy, binder and polymer reinforcement.

The location of the fiduciary markers within the composite material may be relative to a datum, such as a point or plane on the surface of, or within, the material. Alternatively, or additionally, the location of the markers may be relative to one another.

The inclusion of the plurality of fiduciary markers comprising elements which attenuate x-rays to an extent greater than the fiber and resinous material, may be known as “salting” and refers to the inclusion of a limited amount of a material that is insufficient to impact the prime physical properties of the structure (strength, weight etc.).

The term “fiduciary marker” may include an object placed in the field of view of an imaging system which appears in the image produced, for use as a point of reference or a measure. In this context, it may be placed permanently into the imaging subject with an aim of: allowing an enhanced ability to discriminate in the ‘z’ dimension; specifically to enhance sensitivity to delamination as the weave is often perpendicular to the ray path; to provide a permanent map that allows both comparison of the same device over time and for the device to be imaged by imaging sub-components and ‘stitching’ the images together; and, to uniquely and permanently identify that device.

The composite material may be imaged using x-rays to provide unique signature “keys”. These keys may be used both to locate defects within a composite, especially in the depth axis, which may be hard to measure on x-rays; and, may function as a physical unclonable function (PUF) for component verification. For large structures, such as an airplane wing, a single key spanning the entire structure or even one generated from a large area of the structure may not be desirable. Rather a set of keys may be generated from a variety of regions of interest. Such an arrangement may have the added benefit of being able to identify a part even if it has become damaged and broken apart. In this way, the concept of a PUF-per-unit-area may be useful, with signature keys generated from a patch-work of scanned areas. It may also be used to confirm the completeness of coverage.

With regard to being able to check for delamination of composites, it is noted that the problem with the use of x-ray-based detection is that composites are difficult to image as they do not attenuate well, and do not have material variations in attenuation, thus producing low contrast images.

The fiduciary markers may comprise one or more of copper, iron, molybdenum, tungsten and gold. Other elements or compounds may be employed as they provide contrast with the resinous material and fiber when imaged using x-rays.

The fiduciary markers may comprise carbon nanotubes with metallic cores. Other metallic molecules (or other attenuating markers) may be introduced into the resinous material when the composite is being formed at a level that will not negatively impact the functional properties of the device with respect to strength and weight. It is also possible that a carbon nanotube is ‘tagged’ with an attenuating marker. This may be effected by not completing the standard carbon nanotube manufacture process thus leaving a ferrous molecule on the inside of the carbon nanotube. It is also possible to have one or more metal sheaths or metal particle “decorations” on the carbon nanotube. These may result from additional processing steps, such as the application of coatings, etc.

The fiduciary markers may comprise particles having a size of approximately 1 to 40 μm. Other sizes such as in the range 50-5000 nm are contemplated.

The resinous material may comprise approximately less than 0.1% by weight of the fiduciary markers.

The fiduciary markers may be invisible to the naked eye from outside the material.

The ratio of fiduciary markers to resinous material, by volume, may vary through the material to provide an indication of their location. For instance, the ratio may increase or decrease through the material from one side to an opposite side. For example, the ratio may increase or decrease with each layer of material (if the material has been formed in an additive manufacturing manner). Determining the ratio at any given point in the material (by means of x-ray imaging) may provide an indication of location within the material.

The quantity of fiduciary markers within the resinous material may vary in a controlled manner through the material. The term “controlled manner” includes a regular increase/decrease in quantity with position, however, other changes in quantity may be included too, such as a logarithmic increase/decrease, and an increase/decrease controlled by a known algorithm. Determining the quantity at any given point in the material (by means of x-ray imaging) may provide an indication of location within the material.

Likewise, the size and/or composition of the fiduciary markers within the resinous material may vary in a controlled manner through the material. Determining the size and/or composition at any given point in the material (by means of x-ray imaging) may provide an indication of location within the material.

The fiduciary markers may be arranged regularly throughout the resinous material, or at defined intervals on the fiber within a composite. For instance, a regular 2D pattern may be produced in each layer to create an overall 3D pattern. This may more easily assist in determining de-lamination or ply-wrinkling of layered materials.

A method of manufacture of a composite material may comprise the steps of applying resinous material, and a plurality of fiduciary markers, to a fiber, the fiduciary markers comprising elements which attenuate x-rays to an extent greater than the fiber and resinous material such that their location within the composite material is determinable by means of x-ray imaging.

The x-ray system employed allows for digital tomosynthesis, also known as limited-angle tomography, which provides depth information in the form of distinct “slices” through an object. The x-ray system may use a two-dimensional ‘sweep’ to allow enhanced use of super-resolution. The ‘sweep’ means that the distributed source of x-ray emitters is arranged in a 2D plane, as opposed to a 1D line.

The amount of data accessible via the database, to a subscriber of the database, may depend on factors such as the identity of the user, the nature of their need for the data, such as whether it is for the purpose of checking the structural integrity of the product or checking its identity. Access to the database may be sold or licensed. A cloud registration platform (i.e. one remote from the x-ray imaging system) may be employed for key generation.

The composite material may be imaged at the time of manufacture and the unique relative position of the fiduciary markers may be recorded. The absolute position of the fiduciary markers may be compared at test points allowing definitive identification of variation in the structure. The relative position of the fiduciary markers may be unique, allowing an ‘image stitching’ approach to examining a large item, such as a whole aircraft superstructure, using a system with a detector smaller than the device being imaged, but at the same time giving confidence that all of the structure has been imaged.

The presence of the key may enhance the ability to perform longitudinal analysis (an analysis carried out over time) as (for instance) an increased separation of two individual markers, particularly in the ‘z’ dimension may be indicative of damage, such as delamination.

Each item may have a unique key, allowing parties to identify counterfeit products. A relatively large item may include several keys, allowing for the identification of the specific element of a larger structure, say in the event of recovery of fragments following an aircraft crash.

The presence of keys within a structure, item or product may be used to identify its owner if the key has been recorded at the time of sale.

The probing of the item may determine its key, by means of x-ray imaging. The generation of the key may convert the x-ray images into strings in the Hamming space, and the use of “fuzzy discretizers” may allow for the generation of “noise robust vectors”. These vectors, a set of three-dimensional coordinates T ⊂ Z3, may be converted into the unique “keys”. The determination of the keys may require several scans and the verification or matching of these keys in a secure database may require statistical methods that operate in noisy environments. In practice, conversion of the x-ray scans into vector codes may involve pre-processing including filtering (such as Gabor filters), thresholding and sampling the output which may then be encoded using one of several algorithms.

If an item is subjected to 2D x-ray imaging, the location of the fiduciary markers relative to one another may be determined in one plane. If the item is subjected to 3D imaging, the location of the fiduciary markers relative to one another may be determined in more than one plane. This limitation of 2D imaging may be exploited to give a simple means of checking whether or not an item is counterfeit without the need to reveal the 3D key or even allowing access to the 3D key database. In this way, a field inspection for part authenticity may be made without compromising the security of manufactures' ability to validate a part using a 3D scan. A 3D scan may be required for checking structural integrity.

The fiduciary markers may be represented as speckles of a color, different to the color of the resinous material, on the x-ray images.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is flow chart showing a series of steps for producing a composite material and for generating and checking keys;

FIG. 2 is an x-ray image of a composite material;

FIG. 3 is a schematic view of an x-ray imaging system; and

FIG. 4 is an x-ray image of another composite material.

### DETAILED DESCRIPTION

The present invention will be described with respect to certain drawings but the invention is not limited thereto but only by the claims. The drawings described are only schematic and are non-limiting. Each drawing may not include all of the features of the invention and therefore should not necessarily be considered to be an embodiment of the invention. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein. Likewise, method steps described or claimed in a particular sequence may be understood to operate in a different sequence.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Similarly, it is to be noticed that the term “connected”, used in the description, should not be interpreted as being restricted to direct connections only. Thus, the scope of the expression “a device A connected to a device B” should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other. For instance, wireless connectivity is contemplated.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

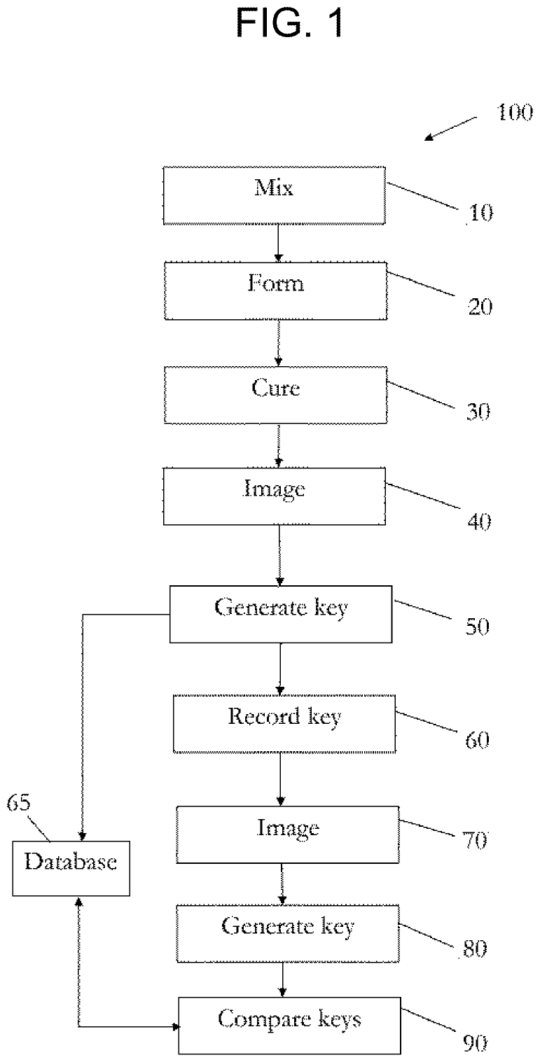

FIG. 1 portrays the basic method steps 100 in a typical process of manufacture including checking the identity and/or structural integrity of the composite material.

In the first step 10, the resinous material is mixed with the fiduciary markers. In the second step 20, the mixed resinous material and fiduciary markers is applied to fibers. A mold may be employed to form a specific shape. The resulting composite material is then cured in the third step 30. Vacuum forming and the application of heat may be employed in the forming and curing steps.

The resulting composite material is then x-ray imaged in the fourth step 40. The x-ray images are then processed in the fifth step 50 to generate a unique key based on the location of the fiduciary markers relative to one another.

This key is then recorded in a database 65 in the sixth step 60.

At this point the key may be compared to a “standard” key, possibly stored in the database to check the integrity of the material. In other words, to check that its structure complies with pre-determined quality control requirements.

At a later time, the composite material may also be x-ray imaged in the seventh step 70. The x-ray images may then be processed in the eighth step 80 to generate a key based on the location of the fiduciary markers relative to one another.

This key may then be compared in the ninth step 90 to various keys stored in the database 65 from the sixth step 60. The comparison may confirm the identity of the composite material or may reveal that it is counterfeit, in that no such key exists. Alternatively, or additionally, the comparison of the later key with a previous key for the same composite material may be used to assess its structural integrity in that the markers are in the same place or have moved indicating failure within the material.

It is to be understood that the material imaged in the seventh step 70 may be different from the material imaged in the fourth step 40. This may allow the determination of the identity of the new material and/or to determine if it is counterfeit.

The key may be a set of co-ordinates of the location of all or some of the fiduciary markers identified in the images.

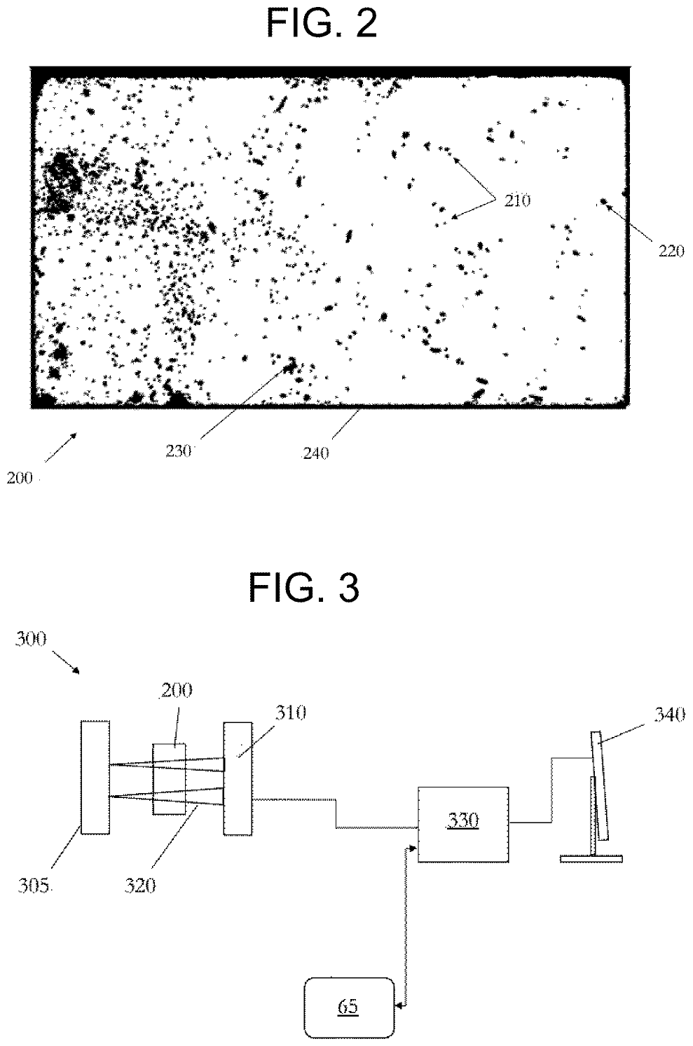

FIG. 2 shows an example of an x-ray image of a composite material 200. Within the image various speckles are visible. Some speckles 210 may relate to the fiduciary markers. Other speckles 220 may relate to material sensitive to ionizing radiation. Further speckles 230 may relate to carbon nanotubes with metallic cores. The location of the markers relative to one another may be determined. Alternatively, and/or additionally, the location of at least some of the markers may be determined relative to a datum, such as the base 240 of the material 200.

An example x-ray imaging system 300 is shown in FIG. 3. It comprises x-ray emitters 305, which may be one or more flat panel arrays, and a detector 310. A composite material 200 is arranged between the two and is subjected to x-rays 320. The resultant images are processed in a processor 330 to generate keys. The processor may be connected to a database 65 for storing images and/or the keys generated therefore. It will be understood that the processor 330 and/or database 65 may be located distal from the x-ray emitters 305 and detector 310.

A monitor 340 is provided for controlling the system 300.

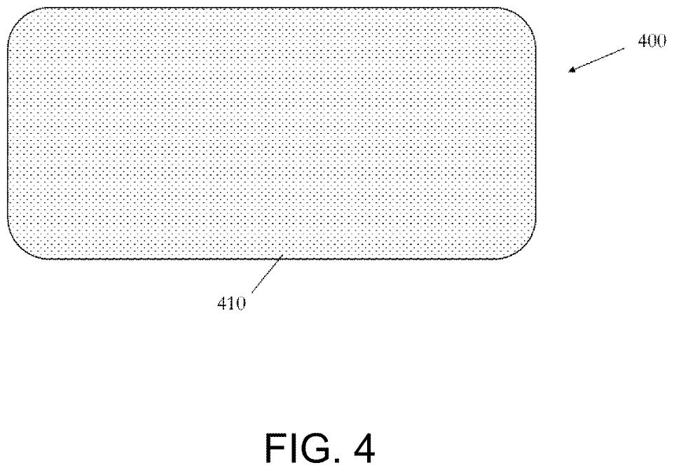

FIG. 4 is a depiction of an example composite material 400 wherein the fiduciary markers 410 are arranged in a regular pattern. This pattern may also be the result of the markers being arranged at defined intervals on a fiber within the material. This view is a 2D slice through the material. It is to be understood that the regular pattern may be arranged in more than one plane through the material.

1. A method of producing 3D tomosynthesis images of at least a portion of a composite material, the composite material including fiber mixed with resinous material, and a plurality of fiduciary markers, the fiduciary markers comprising elements which attenuate x-rays to an extent greater than the fiber and resinous material such that their location within the portion of composite material is determinable by x-ray imaging, the method comprising the step of providing a composite material, providing an array of x-ray emitters and a digital x-ray detector wherein the array of x-ray emitters and the digital x-ray detector are maintained in fixed relation to one another and to the composite material, x-ray imaging at least a portion of the composite material to provide a first set of 3D tomosynthesis images to determine the relative location of at least some of the fiduciary markers with respect to one another, providing a database and storing the relative location of at least some of the fiduciary markers with respect to one another in the database.

2. The method of claim 1, further comprising the step of comparing the relative locations of the at least some of the fiduciary markers with a predetermined set of locations to evaluate the quality of the composite material.

3. The method of claim 1, further comprising the step of x-ray imaging the portion of composite material at a point in time after the initial imaging to provide a second set of 3D tomosynthesis images to determine the relative location of at least some of the fiduciary markers with respect to one another; and comparing the relative locations of the fiduciary markers in the first and second sets of 3D tomosynthesis images to evaluate the occurrence of change in the structural integrity of the portion of composite material.

4. The method of claim 1, further comprising the step of x-ray imaging at least a portion of another composite material to provide another set of 3D tomosynthesis images to determine the relative location of at least some of the fiduciary markers with respect to one another; and comparing the relative locations of the fiduciary markers in the first set and other set of 3D tomosynthesis images to evaluate the identity of the other composite material.

5. The method of claim 4, wherein the step of evaluating the identity of the other composite material includes the step of determining if the other composite material is a counterfeit product.

6. The method of claim 5, wherein the step of evaluating the identity of the other composite material includes the step of interrogating the database.

7. The method of claim 1, further comprising the step of providing 2D x-ray imaging apparatus and x-ray imaging at least a portion of the composite material to provide a 2D x-ray image to determine the relative location of at least some of the fiduciary markers with respect to one another; and comparing the relative locations of the fiduciary markers in the 2D image with the first set of images to evaluate the identity of the other composite material.

8. The method of claim 7, wherein the step of evaluating the identity of the other composite material includes the step of determining if the other composite material is a counterfeit product.

9. The method of claim 7, wherein the step of evaluating the identity of the other composite material includes the step of interrogating the database.

10. The method of claim 1, further including the step of providing a processor and using the processor to determine the relative location of the at least some of the fiduciary markers with respect to one another.

11. The method of claim 1, further including the step of repeatedly moving either or both of the array of x-ray emitters and the digital x-ray detector to a different portion of the composite material for x-ray imaging thereof, so as to x-ray image multiple portions of a composite material, wherein the array of x-ray emitters and the digital x-ray detector are maintained in fixed relation to one another and to the composite material at the time of x-ray imaging.

12. The method of claim 11, further including the step of processing the various sets of x-ray images obtained for each portion of the composite material to create a single set of contiguous images of the composite material.
